(19) United States
(12) Patent Application Publication
(10) Pub. No.: US 2025/0262178 A1
(43) Pub. Date: Aug. 21, 2025

(54) NANOEMULSION COMPRISING DICLOFENAC

(71) Applicant: HALEON CH SARL, Prangins (CH)

(72) Inventors: Maria Jose ALONSO, Galicia Region (ES); Desiree TEIJEIRO, Galicia Region (ES); Ovidio CATANZANO, Galicia Region (ES); German Berrecoso CUNA, Galicia Region (ES)

(21) Appl. No.: 18/570,337

(22) PCT Filed: Jun. 28, 2022
(86) PCT No.: PCT/EP2022/067655

§ 371 Date: Dec. 14, 2023

(30) Foreign Application Priority Data
Jun. 30, 2021 (EP) 21382581.3

(51) Int. Cl. A61K 31/196; A61K 9/00; A61K 9/107; A61K 47/14; A61K 47/18

(52) U.S. Cl. CPC A61K 31/196; A61K 9/0014; A61K 9/1075; A61K 47/14; A61K 47/186

(57) ABSTRACT

A nanoemulsion for topical drug delivery, comprising diclofenac alkali metal salt, diclofenac free acid, a solvent of the glycol, or glycol ether or glycol ether ester family, a nonionic surfactant with a hydrophilic lipophilic balance (HLB) value of about 14-16, caprylic capric triglyceride, and water. The nanoemulsion is useful in the treatment of joint pain, osteoarthritis, muscle pain, back pain and/or inflammation.

### FIELD OF THE INVENTION

This disclosure relates to nano-formulations for the topical delivery of diclofenac. Specifically, it relates to a novel nanoemulsion formulation comprising a combination of diclofenac (free acid) and diclofenac alkali metal salt.

### BACKGROUND TO THE INVENTION

Topical, transdermal products comprising diclofenac, a non-steroidal anti-inflammatory drug (NSAID) of the acetic acid class, are currently available to patients and consumers in Europe and other countries, and are widely used for their analgesic and anti-inflammatory properties, providing an advantage over oral NSAID formulations, inter alia due to reduced systemic adverse effects. However, it is very difficult to develop topical formulations that are pharmaceutically acceptable and at the same time exhibit consumer preferred attributes. Additionally, diclofenac is a particularly challenging ingredient to work with, and poses many challenges to the formulator. Improved topical compositions comprising diclofenac are needed that meet pharmaceutical, technological and consumer expectations.

### SUMMARY OF THE INVENTION

There is provided an oil-in-water nanoemulsion for topical drug delivery, comprising:
a) diclofenac alkali metal salt, in a concentration of about 0.7% to about 1.3% (w/w),
b) diclofenac free acid, in a concentration of about 0.7% to about 1.3% (w/w),
c) a solvent of the glycol, or glycol ether or glycol ether ester family, in a concentration of about 3% to about 7% (w/w),
d) a nonionic surfactant with a hydrophilic lipophilic balance (HLB) value of about 14-16,
e) caprylic capric triglyceride, and
f) water.

In one embodiment, the ratio of diclofenac alkali metal salt to diclofenac free acid is about 0.8:1.2 to about 1.2:0.8.

In one embodiment, the total concentration of diclofenac is from about 1.5% to about 2.5% (w/w).

In one embodiment, the nanoemulsion further comprises: g) a cationic surfactant, in a concentration of about 0.1% to about 0.3% (w/w).

In one embodiment, the nanoemulsion is self-emulsifying.

In one embodiment, the nanoemulsion is transparent.

In one embodiment, the nanoemulsion is alcohol-free.

In one embodiment, component g) is a quaternary ammonium compound.

In one embodiment, component g) is a C₈-C₁₆-alkyl trimethylammonium salt. In one embodiment, component d) is a mixture of polyethylene glycol mono- and diesters of 12-hydroxystearic acid.

In one embodiment, component d) is a mixture of polyethylene glycol mono- and diesters of 12-hydroxystearic acid and comprises about 20% to about 40% (w/w) free polyethylene glycol.

In one embodiment, component e) has a caprylic acid percentage of about 55% to about 60% and a capric acid percentage of about 40% to about 45%.

In one embodiment, component d) is present in a concentration of about 20% to about 40% (w/w) and component e) is present in a concentration of about 2.5% (w/w) to about 7.25% (w/w).

In one embodiment, the nanoemulsion is provided in a container comprising an applicator for direct application onto the skin.

In one embodiment, the nanoemulsion is for use in the treatment of joint pain, osteoarthritis, muscle pain, back pain and/or inflammation.

### DETAILED DESCRIPTION OF THE INVENTION

To facilitate extended therapeutic efficacy, an important objective of the inventors has been to provide a composition which facilitates improved diclofenac penetration through the skin, into regions of interest for anti-inflammatory and anti-nociception effects. The skin, however, is a natural barrier, providing hydrophobic qualities to minimise percutaneous absorption of ionised drugs, such as diclofenac.

One way to improve penetration of diclofenac is through incorporation of higher concentrations of the active pharmaceutical ingredient (API). This however entails numerous difficulties. Higher amounts of API can lead to chemical instability due to a higher or lower pH. Higher amounts of API can also cause instability due to interaction with other ingredients of the formulation. The approach of raising API concentration is especially limited for diclofenac, due to the very limited solubility and high cristallisation tendency of diclofenac. Furthermore, higher API concentrations render products more expensive, and enlarge their environmental footprint. Inter alia for environmental reasons, it is desirable to achieve the highest possible penetration with the least concentration of active. Thereby, API usage could be reduced without compromising efficacy. Furthermore, exposure of the environment with “unused” active could be reduced.

Another way to achieve higher penetration of diclofenac is through incorporation of (higher) concentrations of permeation enhancer(s) in a formulation. However, permeation enhancer(s) can also cause skin irritation, and can, in some concentrations, even have a penetration retarding effect. Penetration enhancers can also lead to instability of multiphase topical systems. Additionally, product characteristics like rheology or appearance can be negatively impacted by inclusion of permeation enhancers. It would be preferred to provide a product that achieves high penetration of diclofenac even without the need to incorporate a permeation enhancer. But ideally, penetrations could nevertheless be successfully incorporated into the formulation to further enhance penetration of the API, without a negative impact on stability, product attributes and safety. Thereby, a product range with different strengths or potencies could be created, based on the base formulation.

Several challenges lie in the manufacture of topical products. Many methods of manufacture are cumbersome, expensive, and waste (water) intensive, e.g. because they require numerous vessels. Another problem specific to multiphase systems is the energy impact of usual emulsification methods. Using (high) shear homogenization, especially for prolonged periods of time, is energy intensive. This leads to higher production cost and can also negatively impact stability because of higher temperatures and oxygen introduction. Therefore, there is a need for a product that can be manufactured in an economic and environmentally friendly method of manufacture, without high energy impact.

An additional challenge is to provide a stable composition suitable for commercial distribution and sale. The minimum stability profile requires overcoming challenges including the resistance to phase separation in multiphase systems, creaming, precipitation, and flocculation.

Another challenge is to provide a topical product with pleasant attributes. Currently, consumers prefer non greasy, light products. Also preferred are clear, transparent, or translucent topical products. Ideally the products should have pleasing spread characteristics, and dry quickly without leaving residue on the skin. The texture of the product needs to be pleasant to consumers. Cooling properties are often preferred, however incorporation of excipients with a cooling effect is also limited due to various formulation challenges.

Consequently, there remains a need for a topical, transdermal diclofenac formulation that addresses one or more of the described challenges.

Unless indicated otherwise, concentrations are given in % (w/w).

The term “about” in relation to a numerical value x means, for example, x±10%, x±5%, x±4%, x±3%, x±2%, x±1%.

### Pharmaceutical Compositions

The compositions disclosed herein are compositions for topical drug delivery. This means, the compositions comprise an active pharmaceutical ingredient (API). The compositions are designed to be applied on skin of a patient who is in need of treatment with that API. The compositions are effective vehicles for the API to permeate skin. The compositions disclosed herein allow for the API to permeate through skin and to a site of action. The site of action may for example be a joint. Permeation of the API from a composition and through the skin can be determined in sophisticated pre-clinical experiments, such as the experiments described later herein in the experimental section. Skin is a complex structure comprising several tissue layers. Therefore, the skin is a significant barrier for APIs. The compositions of the present disclosure are not intended for treatment of dermal conditions. In one embodiment, the compositions are not for the treatment of dermal conditions.

This disclosure provides pharmaceutical compositions for topical drug delivery which are nanoemulsions with an aqueous outer phase. Nanoemulsions are disperse systems that comprise nano-sized droplets which are dispersed in an outer, continuous, aqueous phase. The droplets comprise a lipophilic core, often referred to as inner phase, surrounded by amphiphilic molecules called surfactants. The droplets of inner phase and surfactants are called micelles. The micelles have an average size below about 100 nm, usually determined by dynamic light scattering (DLS) technique. The micelles might therefore also be referred to as nanomicelles.

The nanoemulsions described herein comprise nanomicelles, the micelles comprising active pharmaceutical ingredients (APIs), a surfactant, a solvent, and an oil. Charged, hydrophilic moieties, often called head groups, of components in the nanomicelles are oriented towards the outer aqueous phase, whilst uncharged, lipophilic moieties are directed towards the core of the nanomicelles.

Nanoemulsions are very well suited for the formulation of tailored pharmaceutical compositions for topical drug delivery. Additional ingredients may be incorporated within the core of the nanomicelles, in an area close to the charged head groups of the complex, at an interphase between the nanomicelle and the outer phase, or in the aqueous phase. Where an ingredient will accumulate depends on the exact composition, and the characteristics of the additional ingredient, such as hydrophilic lipophilic balance (HLB), partition coefficient and polarity. However, incorporating an additional ingredient, and even changing the concentration of an ingredient, can also destabilise the system, leading to flocculation, emulsion breaking, coalescence from nano-micelles to larger droplets, creaming or the like. Therefore, nanoemulsions are a very delicate pharmaceutical form.

In one embodiment, the compositions are nanoemulsions consisting of a disperse oil phase in an aqueous continuous phase. In one embodiment, the compositions comprise an aqueous outer phase. In one embodiment, the compositions are biphasic. In one embodiment, the compositions are oil-in-water nanoemulsions. In one particular embodiment, the compositions are oil-in-water nanoemulsions and do not comprise an external oil phase.

The compositions disclosed herein comprise a combination of diclofenac alkali metal salt (component a)) and diclofenac free acid (component b)) as the active pharmaceutical ingredients (APIs). Diclofenac sodium and diclofenac potassium salts are especially useful. Most preferred is diclofenac sodium.

Preferably, diclofenac and diclofenac alkali metal salt are the only API in the compositions. In one embodiment, the ratio of diclofenac alkali metal salt to diclofenac free acid is about 0.8:1.2 to about 1.2:0.8 (w: w). In another embodiment, the ratio is about 1:1 (w: w). In one embodiment, the diclofenac alkali metal salt is present in a concentration of about 0.7% to about 1.3% (w/w). In another embodiment, the diclofenac free acid is present in a concentration of about 0.7% to about 1.3% (w/w). In one embodiment, the nanoemulsion comprises diclofenac free acid in a concentration of about 1% (w/w) and diclofenac sodium salt in a concentration of about 1% (w/w). Diclofenac alkali metal salt dissociates in solution forming a diclofenac anion and an alkali metal cation. The diclofenac anion is important for the formation of the nanomicelles. The diclofenac free acid can accumulate in the core of the nanomicelle, thereby raising the overall content of diclofenac in the composition, surprisingly without hampering or destabilising the formation of the nanomicelles and the overall composition.

The nanoemulsion of this disclosure comprises a solvent (component c)). The solvent is a non-volatile solvent. Preferably, the solvent is a solvent of the glycol, or glycol ether or glycol ether ester family. Preferably, the solvent has a boiling point of above about 150° C. at normal pressure. Preferably, the solvent has an octanol/water partition coefficient expressed as log P_{ow} of about −0.5 to about 1 at 20° C.

In one embodiment, the nanoemulsion comprises diethylene glycol monoethyl ether, diethylene glycol monomethyl ether, or a mixture thereof. In one embodiment, the solvent is diethylene glycol monoethyl ether. The solvent is able to solubilise the APIs, preventing crystallisation.

In one embodiment, the solvent is present in a concentration of about 3% to about 7% (w/w). In a preferred embodiment, the solvent is present in a concentration of about 5% (w/w).

The nanoemulsion compositions can optionally comprise a cationic surfactant (component g)). The cationic surfactant may have one positive charge per molecule or may have multiple positive charges per molecule. Preferably, the cationic surfactant has one positive charge per molecule.

The cationic surfactant is a cationic oil-in-water surfactant. In one embodiment, the cationic surfactant is a quaternary ammonium compound. In one embodiment, the cationic surfactant is an alkyl trimethylammonium or a dialkyl dimethylammonium salt or a combination thereof. Preferred are alkyl trimethylammonium salts. In one embodiment, the cationic surfactant is selected from the group of benzalkonium salt, benzethonium salt, cetylalkonium salt, cetylpyridinium salt, cetyltrimethylammonium salt, dequalinium salt, cetrimide and combinations thereof. In a preferred embodiment, the cationic surfactant is a C₈ to C₁₆-alkyl trimethylammonium salt. More preferred is C₁₁ to C₁₃ trimethylammonium salt. In a preferred embodiment, the cationic surfactant is cetrimide.

In one embodiment, the cationic surfactant is present in a concentration of about 0.1% to about 0.3% (w/w).

The nanoemulsion of this disclosure comprises a combination of non-ionic surfactant and a specific oil (components d) and e)). The nonionic surfactant is involved in the formation of the nanomicelles in the nanoemulsion.

In one embodiment, the nanoemulsion comprises a nonionic surfactant with a hydrophilic lipophilic balance (HLB) value of about 14-16.

In one embodiment, the nonionic surfactant comprises a polyethylene glycol ester of C₁₄ to C₁₈ fatty acids.

In one embodiment, the nonionic surfactant is a mixture of polyethylene glycol mono- and diesters of 12-hydroxystearic acid comprising about 20% to about 40% (w/w) free polyethylene glycol. Alternatively, the nonionic surfactant can be Macrogol-15-Hydroxystearate.

In one embodiment, the nonionic surfactant is present in a concentration of about 20% to about 40% (w/w). In another embodiment, the nonionic surfactant is present in a concentration of about 25% to about 35%/w/w). In another embodiment, the nonionic surfactant is present in a concentration of about 30% (w/w).

The oil is caprylic capric triglyceride. The oil has a high solubilisation capacity for diclofenac.

In one embodiment, the oil is caprylic capric triglyceride with a caprylic acid percentage of about 55% to about 60% and a capric acid percentage of about 40% to about 45%. It has been found that the specific ratio of fatty acids provides for stable nano-micelles. This fatty acid composition of the used oil has been found provide the most stable formulations, with the least tendency of phase separation.

In one particular embodiment, the oil is Labrafac Lipophile WL1349. Labrafac Lipophile WL1349 is a trade name and means a mixture of medium-chain triglycerides. It consists of medium-chain triglycerides of caprylic (C₈) and capric (C₁₀) acids. It has a caprylic acid percentage of about 55% to about 60% and a capric acid percentage of about 40% to about 45%. It has a viscosity of from about 25 to about 33 mPa·s at 20° C. It has a HLB value of about 1 In one embodiment, the oil is a mixture of medium-chain triglycerides. In one embodiment, the oil comprises medium-chain triglycerides of caprylic (C₈) and capric (C₁₀) acids. In one embodiment, the oil consists of medium-chain triglycerides of caprylic (C₈) and capric (C₁₀) acids. In one particular embodiment, the oil has a caprylic acid percentage of about 55% to about 60% and a capric acid percentage of about 40% to about 45%.

In one embodiment, the oil is present in a concentration of about 2.5% to about 7.25% (w/w). In another embodiment, the oil is present in a concentration of about 4% to about 6% (w/w). In another embodiment, the oil is present in a concentration of about 5% (w/w).

The outer phase of the nanoemulsions is f), water. It may be pharma grade water or purified water or any other water suitable for pharmaceutical compositions. In one embodiment, the nanoemulsions comprises a concentration of at least about 50% (w/w) water. In another preferred embodiment, the nanoemulsions comprises a concentration of about 50% to about 60% (w/w) water. The outer phase can optionally comprise additional hydrophilic ingredients.

In one embodiment, the nanoemulsion does not contain a volatile alcohol, such as ethanol or isopropanol. C₂ to C₃ alcohols are often incorporated into topical pharmaceutical compositions to increase the solubilisation capacity of the composition for the API. However, these agents may not be preferred ingredients because of their potential for skin irritation. Furthermore, volatile alcohols may solubilise the APIs well, however they evaporate quickly upon application onto the skin, which can lead to cristallisation of the API on the skin. Preferably, the APIs are in fully solubilised state in the disclosed nanoemulsions, without the need to incorporate an alcohol.

Preferably, the nanoemulsion is translucent or transparent. In one preferred embodiment, the nanoemulsion is transparent. Translucent or transparent compositions are often preferred by patients and can lead to a better compliance. Nanoemulsions with an average micelle size, determined by dynamic light scattering, of about 10 nm to about 50 nm, can be regarded as translucent. Nanoemulsions with an average micelle size, determined by dynamic light scattering, of about 5 nm to 10 nm, can be regarded as transparent. Average micelle size is the average size of the micelles present in the nanoemulsion, determined by dynamic light scattering.

The nanoemulsions may comprise further excipients, such as perfumes, chelating agents, preservatives, antioxidants, or the like.

Preferably, the nanoemulsion is self-assembling. In other words, surfactant molecules in the described composition aggregate into a micelle structure upon mixture of the ingredients. This process involves complex interfacial hydrodynamic phenomena and depends on the system composition properties. Only very specific pharmaceutical excipient combinations and concentrations lead to efficient self-assembly into nanoemulsions. Self-assembling formulations are preferred because they are more stable, less susceptible of phase separation, creaming, flocculation, and the like. They are also preferred because these do not require high energy manufacturing methods like micro fluidization, high pressure homogenization, ultrasonic treatment, or the like.

Preferably, the compositions have a pH suitable for topical administration. Preferably, the pH is in the range of about 8 to about 8.5. Preferably, the pH is inherent to the compositions without addition of an acid, base or buffer.

### Methods of Manufacture

The compositions of this disclosure may be prepared as follows. Components a) to e) and optionally g) are weighed into a vessel and mixed (about 700 rotations per minute), for about 30 minutes at about 40° C. to form a homogenous mixture.

Component f), water, is added under stirring at about 700 rpm for about 30 minutes at about 40° C.

### Methods of Treatment

The nanoemulsions may be administered to mammals suffering from joint pain, osteoarthritis, muscle pain, back pain and/or inflammation. The nanoemulsions are useful in the treatment of joint pain, osteoarthritis, muscle pain, back pain and/or inflammation. The methods of treatment can comprise administering to a subject in need thereof a pharmaceutically effective amount of the nanoemulsion disclosed herein. The method can comprise administering the nanoemulsion to skin that covers a body part that suffers from or causes one or more of joint pain, osteoarthritis, muscle pain, back pain and/or inflammation. The method can comprise rubbing in until the skin feels dry. The method can comprise applying the nanoemulsion 2-4 times per day. Alternatively, they may be applied twice per day. Typical doses of the nanoemulsion comprise 0.09 mg diclofenac equivalent/cm² (2-4 times per day application) or 0.21 mg diclofenac equivalent/cm² (twice per day application).

The nanoemulsions may be provided in a container comprising an applicator for direct application onto the skin. The nanoemulsions may be provided in a package comprising a means for dosing. In one embodiment, the dosing means is a pump. In one embodiment, the means for dosing is a pipette.

### Clauses

A nanoemulsion for topical drug delivery, comprising:
a) diclofenac alkali metal salt, in a concentration of about 0.7% to about 1.3% (w/w),
b) diclofenac free acid, in a concentration of about 0.7% to about 1.3% (w/w),
c) diethylene glycol monoethyl ether, in a concentration of about 3% to about 7% (w/w),
d) a mixture of polyethylene glycol mono- and diesters of 12-hydroxystearic acid comprising about 20% to about 40% (w/w) free polyethylene glycol,
e) caprylic capric triglyceride with a caprylic acid percentage of about 55% to about 60% and a capric acid percentage of about 40% to about 45%, and
f) water.

In one embodiment, the ratio of diclofenac alkali metal salt to diclofenac free acid is about 0.8:1.2 to about 1.2:0.8 (w:w).
3. In one embodiment, the total concentration of diclofenac is from about 1.5% to about 2.5% (w/w).
4. In one embodiment,, the nanoemulsion further comprises g) a cationic surfactant, in a concentration of about 0.1% to about 0.3% (w/w).
5. In one embodiment, the nanoemulsion is self-emulsifying.
6. In one embodiment, the nanoemulsion is transparent.
7. In one embodiment, the nanoemulsion is alcohol-free.
8. In one embodiment, component g) is a quaternary ammonium compound.
9. In one embodiment, component g) is a C₈-C₁₆-alkyl trimethylammonium salt.
10. In one embodiment, component d) is a mixture of polyethylene glycol mono- and diesters of 12-hydroxystearic acid.
11. In one embodiment, component e) is Labrafac Lipophile WL1349.
12. In one embodiment, component d) is present in a concentration of about 20% to about 40% (w/w) and component e) is present in a concentration of about 2.5% to about 7.25% (w/w).
13. In one embodiment, the nanoemulsion is for use in the treatment of joint pain, osteoarthritis, muscle pain, back pain and/or inflammation.
14. In one embodiment, the composition is provided in a container comprising an applicator for direct application onto the skin.

### EXAMPLE

TABLE 1
Formulation of exemplary nanoemulsion

| Ingredient | Concentration [% w/w] |
|---|---|
| Diclofenac sodium | 1.074 |
| Diclofenac free acid | 1 |
| Labrafac WL1349 | 5 |
| Kolliphor HS15 | 30 |
| Transcutol P | 5 |
| Cetrimide | 0.25 |
| Ultrapure water | q.s. up to 100 |

The formulation was prepared as follows:

The diclofenac sodium diclofenac free acid, Labrafac WL1349, Transcutol P, Kolliphor HS15 and Cetrimide were weighed into a glass vial. The vial was heated to 40° C. under continuous stirring, with a 10nm magnetic stirring bar, in a magnetic heat stirrer, during 30 minutes at 700 rpm until a homogenous solution is formed. The water was weighed into a separate vial and added at once under stirring with 700 rpm at 40° C. Stirring was continued for 30 minutes at the same temperature. The vial was closed with a cap during the stirring.

Embodiments described herein can be understood more readily by reference to the detailed description and examples. Elements and methods described herein, however, are not limited to the specific embodiments presented in the detailed description and examples. It should be recognized that the exemplary embodiments herein are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

It is further to be understood that the feature or features of one embodiment may generally be applied to other embodiments, even though not specifically described, or illustrated in such other embodiments, unless expressly prohibited by this disclosure or the nature of the relevant embodiments. Likewise, compositions and methods described herein can include any combination of features and/or stops described herein not inconsistent with the objectives of the present disclosure. Numerous modifications and/or adaptations of the compositions and methods described herein will be readily apparent to those skilled in the art without departing from the present subject matter.

1. An oil-in-water nanoemulsion for topical drug delivery, comprising:
a) diclofenac alkali metal salt, in a concentration of about 0.7% to about 1.3% (w/w),
b) diclofenac free acid, in a concentration of about 0.7% to about 1.3% (w/w), c) a solvent of the glycol, or glycol ether or glycol ether ester family, in a concentration of about 3% to about 7% (w/w),
d) a nonionic surfactant with a hydrophilic lipophilic balance (HLB) value of about 14-16,
e) caprylic capric triglyceride, and
f) water.

2. A nanoemulsion according to claim 1, wherein the ratio of diclofenac alkali metal salt to diclofenac free acid is about 0.8:1.2 to about 1.2:0.8 (w:w).

3. A nanoemulsion according to claim 1, wherein the total concentration of diclofenac is from about 1.5% to about 2.5% (w/w).

4. A nanoemulsion according to claim 1, further comprising
g) a cationic surfactant, in a concentration of about 0.1% to about 0.3% (w/w).

5. A nanoemulsion according to claim 1, which is self-emulsifying.

6. A nanoemulsion according to claim 1, which is transparent.

7. A nanoemulsion according to claim 1, wherein the nanoemulsion is alcohol-free.

8. A nanoemulsion according to claim 4, wherein component g) is a quaternary ammonium compound.

9. A nanoemulsion according to claim 8, wherein component g) is a C₈-C₁₆-alkyl trimethylammonium salt.

10. A nanoemulsion according to claim 1, wherein component d) is a mixture of polyethylene glycol mono- and diesters of 12-hydroxystearic acid.

11. A nanoemulsion according to claim 10, wherein d) comprises about 20% to about 40% (w/w) free polyethylene glycol.

12. A nanoemulsion according to claim 1, wherein component e) has a caprylic acid percentage of about 55% to about 60% and a capric acid percentage of about 40% to about 45%.

13. A nanoemulsion according to claim 1, wherein component d) is present in a concentration of about 20% to about 40% (w/w) and component e) is present in a concentration of about 2.5% to about 7.25% (w/w).

14. A nanoemulsion according to claim 1, provided in a container comprising an applicator for direct application onto the skin.

15. A nanoemulsion according to claim 1, for use in the treatment of joint pain, osteoarthritis, muscle pain, back pain and/or inflammation.
